(19) United States
(12) Patent Application Publication
(10) Pub. No.: US 2020/0397526 A1
(43) Pub. Date: Dec. 24, 2020

(54) INFANT CAR SEAT CHALLENGE TESTING STATION

(71) Applicant: ARTi, Incorporated, Hampton, VA (US)

(72) Inventor: Rosemary Evans Williams, Suffolk, VA (US)

(21) Appl. No.: 16/449,656

(22) Filed: Jun. 24, 2019

(51) Int. Cl. A61B 50/13; B62B 3/00; B62B 3/04

(52) U.S. Cl. CPC A61B 50/13; B62B 3/005; B62B 5/08; A61G 2200/14; B62B 3/04

(57) ABSTRACT

An infant car seat challenge testing station. The testing station has a car seat holding area with means to secure straps to the holding area and an infant car seat. The testing station has a bracket to which monitoring equipment may be secured, the bracket being moveably attached to the testing station. The testing station further has storage areas and wheels for mobility.

### TECHNICAL FIELD

The present technology is related to car seat testing for infants prior to discharge from a hospital or other medical facility. More particularly, the present invention relates to medical carts having a work platform that is specifically designed to test infant responses in vehicle car seats.

### BACKGROUND

Car seats reduce mortality and morbidity associated with automobile accidents in infants and children and are required throughout the United States and in other countries. Car seats should always be used, including the initial automobile ride. Commercially available infant seats offer the optimum protection for healthy babies born at term. There are some children who many need special care beds, however, which provide protection for special needs infants. Hypoxemia related to positioning and apneic episodes are common in infants born before term in whom apnea may persist up to and beyond term gestation. Pre-discharge respiratory recordings in very low birth weight infants may reveal significant apnea in many babies otherwise ready for discharge. The use of car seats is recommended for all babies during automobile travel, but this may expose preterm infants to other potential risks.

The American Academy of Pediatrics (AAP) recommends that all preterm neonates undergo a period of observation in a car safety seat before discharge, known as the Infant Car Seat Challenge (ICSC), to monitor for respiratory immaturity and the risk of adverse cardiopulmonary events in the upright position. This observation in a car seat before discharge is designed to monitor for possible apnea, bradycardia or oxygen desaturation.

Car seat testing is usually done in the hospital within one week before your baby is ready to go home. The infant will be placed in a car seat (preferably the car seat brought in by the parents that will actually be used to transport the infant). The infant will remain seated in the car seat for 90 to 120 minutes or the length of the car ride home, whichever is longer. During the test, the infant's heart rate, breathing and oxygen level are monitored in accordance with the guidelines of the ICSC.

There is currently no device configured specifically to meet the needs of care givers testing a child during the ICSC.

In the medical community, carts are configured to store, carry, and transport medical supplies, equipment, materials, and other items in a variety of different patient settings (e.g., doctor's office, hospital, etc.). And while there are protocols and procedures in place to test children and especially small infants but there are obstacles to providing testing that accommodates both the medical needs of the children and the need to provide adequate and comprehensive ICSC testing.

There is a need, therefore, to provide a means to assess children and infants restricted to child car seats in order to adequately assess the ability of children with special or defined needs to the rigors of transport through various means; primarily in cars and other personal vehicles but other means of transport are directly impacted by the need to safely and securely transport children and infants.

What is needed is a medical cart that accommodates testing and analysis of the ability of a child or infant to be transported by conventional means (i.e., by utilizing a child seat) without compromising the health and wellbeing of the child.

The present invention provides for a medical cart specifically constructed to provide both medical care and an optimal means for performing ICSC testing. Such an invention would allow portability so that the testing cart can be brought to the child's location, thus avoiding unnecessary moving of at-risk children, as well as providing the convenience of portable equipment for the task.

### BRIEF DESCRIPTION OF THE DRAWINGS

FIG. 1 is an oblique view of medical cart configured in accordance with an embodiment of the present technology.

FIG. 2 is a side view of medical cart configured in accordance with an embodiment of the present technology.

FIG. 3 is a front view of a medical cart configured in accordance with an embodiment of the present invention.

FIG. 4 is a rear view of a medical cart configured in accordance with an embodiment of the present invention.

FIG. 5 is a top view of a medical cart configured in accordance with an embodiment of the present invention.

### DETAILED DESCRIPTION

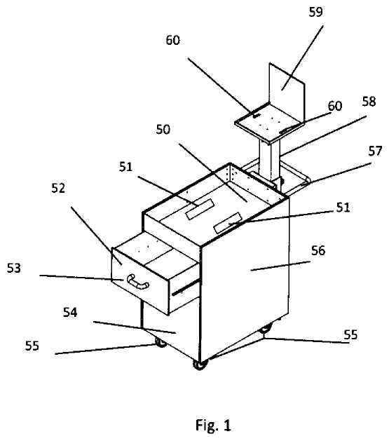

The following disclosure describes various types of medical carts and associated apparatuses and methods. Certain details are set forth in the following description and FIGS. 1-5 to provide a thorough understanding of various embodiments of the disclosure. Other details describing well-known structures and systems often associated with medical carts, however, are not set forth below to avoid unnecessarily obscuring the description of the various embodiments of the disclosure.

Many of the details and features shown in the Figures are merely illustrative of particular embodiments of the disclosure. Accordingly, other embodiments can have other details and features without departing from the spirit and scope of the present disclosure. In addition, those of ordinary skill in the art will understand that further embodiments can be practiced without several of the details described below. Furthermore, various embodiments of the disclosure can include structures other than those illustrated in the Figures and are expressly not limited to the structures shown in the Figures. Moreover, the various elements and features illustrated in the Figures may not be drawn to scale.

In the Figures, identical reference numbers identify identical or at least generally similar elements. To facilitate the discussion of any particular element, the most significant digit or digits of any reference number refer to the Figure in which that element is first introduced. For example, element 50 is first introduced and discussed with reference to FIG. 1.

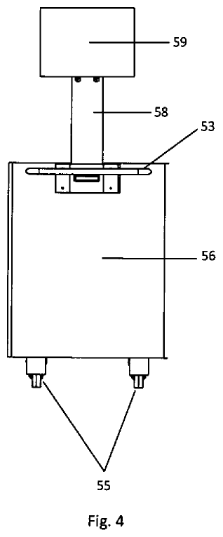

FIG. 1, shows an embodiment of an infant car seat challenge testing station (the “station”) having a car seat holding area 50, the car seat holding area 50 further comprising one or more holes 51, the one or more holes 51 being configured to allow restraining straps be passed through the one or more holes 51. The restraining straps may be chosen from restraining straps that are well known in the art, such as straps with buckles or other mechanical tightening means, straps comprised of hook-and-loop connectors, and the like. The station has a front panel 54, side panels 56, and a back panel (FIG. 4, 61). The station as shown further comprises one or more storage compartments 52 disposed within the front panel 54, as shown comprising a drawer with a handle 53. It will be understood, however, that the one or more storage compartments, while shown with a drawer, may be open storage recesses, open storage recesses with doors or other covering means, or other configurations that are well known in the art. The station further comprises wheels 55. In a preferred embodiment, the wheels 55 will be chosen from the group of wheels that include locking mechanisms so that the station can be locked in place while in use. The station as shown also comprises a handle 57 to be used to maneuver the station. A bracket 59 is also provided to hold monitoring equipment (not shown), which further includes one or more holes 60 to receive one or more straps (not shown) to secure monitoring equipment when in use. It will be understood that the use of the one or more holes 51 in the car seat holding area 50 in conjunction with straps allows the station to be used with various designs of infant car seats. It will also be understood that the use of the one or more holes 60 in the bracket 59 allows the station to be used with various types of monitoring equipment.

The bracket 59 is moveably attached to the station with a telescoping arm 58. Telescoping arms are well known in the art, and it will be understood that various types and designs of telescoping arms may be utilized to attach the bracket 59 to the station without deviating from the scope and intent of the invention as described herein. For example, a telescoping arm may be chosen that moves vertically (as shown) in relation to the station, an articulated arm that provides ranges of motion for the bracket 59 in relation to the station, or other well known means.

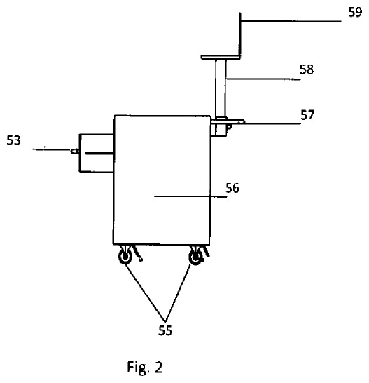

FIG. 2 shows a side view of an embodiment of the present invention, detailing the configuration of the telescoping arm 58 affixed to the station and the bracket 59. As shown the telescoping arm 58 is fully extended up. The drawer 52 is also shown partially disposed within the station.

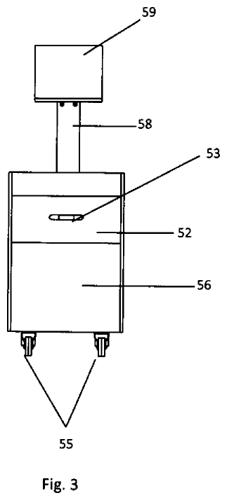

FIG. 3 shows a front view of an embodiment of the present invention, and FIG. 4 shows of view of an embodiment of the present invention as seen from the back.

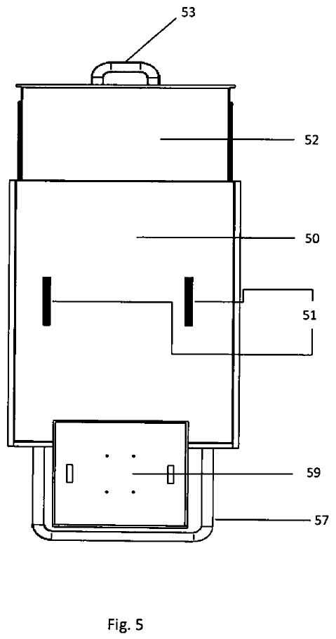

FIG. 5 shows a top-down view of an embodiment of the present invention. As shown, the car seat holding area 50 has two holes 51 through which straps or other restraining means (not shown) may be disposed to facilitate restraining of an infant car seat during testing. The bracket 59 is also shown, as is the drawer 52 partially disposed within the station.

The invention as described herein is not intended to be limiting, as there are modifications which may be made to the invention that remain within the intended, described, and claimed scope of the invention.

We claim:

1. an infant car seat challenge testing station comprising:
a car seat holding area;
the car seat holding area further comprising one or more holes, the one or more holes being configured to allow restraining straps to be passed through the one or more holes;
a front panel;
side panels; and
a back panel.

2. The infant car seat challenge testing station further comprising one or more storage compartments disposed within the front panel.

3. The infant car seat challenge testing station of claim 2 wherein the one or more storage compartments are one or more drawers.

4. The infant car seat challenge testing station of claim 1 further comprising wheels.

5. The infant car challenge testing station of claim 1 further comprising a bracket including one or more holes to receive one or more straps to secure monitoring equipment and wherein the bracket is moveably attached to the infant car seat challenge testing station with a telescoping arm.

6. An infant car seat challenge testing station comprising:
a car seat holding area;
the car seat holding area further comprising one or more holes, the one or more holes being configured to allow restraining straps to be passed through the one or more holes;
a front panel;
side panels;
a back panel;
one or more storage compartments disposed within the front panel;
wheels; and
a bracket including one or more holes to receive one or more straps to secure monitoring equipment and wherein the bracket is moveably attached to the infant car seat challenge testing station with a telescoping arm.
